(19) United States
(12) Patent Application Publication
(10) Pub. No.: US 2023/0077461 A1
(43) Pub. Date: Mar. 16, 2023

(54) WIRELESS IMPLANTABLE ELECTRODE ARRAY

(71) Applicant: University of Southern California, Los Angeles, CA (US)

(72) Inventors: Constantine Sideris, Pasadena, CA (US); Andreas F. Molisch, Los Angeles, CA (US); Ellis Meng, La Canada Flintridge, CA (US)

(21) Appl. No.: 17/931,064

(22) Filed: Sep. 9, 2022

Related U.S. Application Data
(60) Provisional application No. 63/243,056, filed on Sep. 10, 2021.

(51) Int. Cl. A61B 5/294; A61B 5/268; A61B 5/00; H02J 50/00; H02J 50/30

(52) U.S. Cl. CPC A61B 5/294; A61B 5/268; A61B 5/0031; A61B 5/0004; H02J 50/001; H02J 50/30; A61B 2562/046; A61B 2560/0219

(57) ABSTRACT

A flexible implantable electrode array is disclosed, comprising: a shank formed from a flexible polymer material. In an example embodiment, the shank comprises: a waveguide; and a number of chipsets disposed in the shank along the length of the shank, wherein each chipset is configured to measure neural activity in tissue surrounding the shank near the respective chipset, and to communicate signals representative of the measured neural activity via the waveguide. A method for powering and receiving neuronal information from a flexible implantable electrode array comprises: wirelessly communicating power and commands from a backplane to a plurality of chipsets disposed along the length of a shank via a waveguide disposed within the shank; monitoring neural activity proximate each chipset and sending a signal representative of said neural activity from the corresponding chipset transceiver to the backplane via the waveguide.

### CROSS-REFERENCE TO RELATED APPLICATIONS

This application claims the benefit and priority of U.S. Provisional Application No. 63/243,056, entitled “WIRELESS IMPLANTABLE ELECTRODE ARRAY,” filed on Sep. 10, 2021, the entire disclosure of which is hereby incorporated by reference herein in its entirety.

### FIELD

Disclosed herein are apparatus, methods, and/or systems for implantable electrode arrays for gathering and communicating neural information having features of being provided in a flexible shank comprising specially constructed chipsets, and wherein the shank and chipsets are configured to be powered and communicate neural information wirelessly.

### BACKGROUND

Brain machine interfaces (BMI) offer tremendous potential for both basic science and improving quality of life in individuals with brain injury or disease by enabling direct recording (and in some cases stimulation) of the electrical activity of neurons in the brain. Although EEG and ECOG are less invasive methods for acquiring activity of populations of neurons, the activity of individual neurons can only be resolved using implantable electrode arrays (IEAs). IEAs can locally measure action potentials from many different neurons deep inside the brain by using densely packed electrodes.

Recent advances in IEAs have enabled very high density recording concurrently across multiple layers of the brain by using silicon-based multi-electrode arrays and the integration of silicon complementary metal oxide semiconductor (CMOS) technology to embed active recording, amplification, and multiplexing circuitry local to each recording site and arrayed along the length of the implanted shank. Compared to traditional IEAs, which require wires routing the electrical signals from each electrode to external recording circuitry, the silicon CMOS-based technology significantly increases the amount of data which can be recorded.

Unfortunately, due to their rigid and nonconformal nature, the silicon based IEAs cause a significant amount of brain tissue damage both during implantation and also during use as the electrodes shear through tissue as the brain moves. Aside from brain injury, the tissue damage induced by the electrode shank leads to the formation of insulating scar tissue which results in progressive signal deterioration and eventually complete failure in very short timespans of days to months. Furthermore, long silicon IEAs are brittle and thus impractical for deep brain recording. It is, therefore, desired that an implantable electrode array and method of using the same be developed in a manner that will enable ultra-high throughput deep-brain neural recording and do so in a manner that reduces or eliminates brain tissue damage during implantation and use.

### BRIEF DESCRIPTION OF THE DRAWINGS

Other apparatus, systems, methods, features, and advantages of the present invention will be or will become apparent to one of ordinary skill in the art upon examination of the following figures and description. Additional figures are provided in the accompanying Appendix and described therein.

FIG. 1 illustrates a flexible implantable electrode array in an example embodiment, and application of said flexible implantable electrode array to measure neural signals in a brain;

FIG. 2 is a schematic diagram of a flexible implantable electrode array, in accordance with an example embodiment; and

FIG. 3 is an example method for measuring neural signals.

### SUMMARY

In accordance with an example embodiment, a flexible implantable electrode array is disclosed, comprising: a shank formed from a flexible polymer material. In an example embodiment, the shank comprises: a waveguide; and a number of chipsets disposed in the shank along the length of the shank, wherein each chipset is configured to measure neural activity in tissue surrounding the shank near the respective chipset, and to communicate signals representative of the measured neural activity via the waveguide.

In accordance with another example embodiment, a flexible implantable electrode array for receiving and communicating neuronal information is disclosed comprising: a shank formed from a flexible polymer material and having a length greater than a width, wherein the shank comprises a waveguide and a sheath surrounding the waveguide; a waveguide liner disposed in the shank and interposed between the waveguide and the sheath, the waveguide liner formed from a metallic material and extending along a length of the waveguide; and an array of chipsets comprising a number of chipsets positioned serially along the length of the shank, wherein each chipset includes an electrode site that is exposed along a surface of the shank, wherein each chipset communicates signals measured at the electrode site via the waveguide.

In accordance with another example embodiment, a method for powering and receiving neuronal information from a flexible implantable electrode array is disclosed. The method, in an example embodiment, comprises: wirelessly communicating power and commands from a backplane to a plurality of chipsets disposed along the length of a shank via a waveguide disposed within the shank, wherein the shank is formed form a flexible polymer material; monitoring neural activity proximate each chipset via a chipset electrode, and sending a signal representative of said neural activity to a corresponding chipset transceiver within the respective chipset; and wirelessly communicating a radio frequency signal representative of said neural activity from the corresponding chipset transceiver to the backplane via the waveguide.

### DESCRIPTION

Disclosed herein is a flexible implantable electrode array (IEA) that has been specially developed for use in conducting ultra-high throughput deep-brain neural recording in a manner that avoids brain tissue damage associated with the use of conventional IEAs. In an example, the flexible IEA is provided in the form of a flexible shank that is formed from a polymer material such as polymers compatible with microfabrication. In an example, the polymer material is Parylene C. Other example polymer materials include polyimide, SU-8, and the like. In an example, the shank has an elongated configuration and comprises an array of electrodes in the form of chipsets that are arranged along the length of the shank. In an example, the chipsets comprise silicon complementary metal oxide semiconductor (CMOS) chips that are specially configured to be powered and to communicate neural information wirelessly from the shank. In an example, the shank comprises a core and a sheath or layer surrounding the core.

A waveguide is disposed in the shank for purposes of transferring energy to the chipsets (i.e., to power the chipsets) from an energy source external from the shank, and to provide a radio frequency signal from a radiation source external from the shank for enabling the chipsets to modulate the same for communicating neural information from the chipsets to a receiver remote from the shank. In an example, the waveguide is created by placing a layer of material interposed between the shank core and sheath, and is formed from a metallic material. In an example, the metallic material is gold. In another example, the waveguide is created by using materials with different dielectric properties in the core and sheath.

In an example, the radio frequency provided to the chipsets is in the mm-wave or terahertz frequency range (30 to 1000 GHz frequency), and the energy provided to the chipsets is light in the red or infrared part of the light spectrum (e.g., 750 nm wavelength). In another example, the energy provided is in the mm-wave or terahertz frequency range. In an example, the chipsets each comprise a first chip that is attached back-to-back with a second chip, wherein the first chip is configured to receive the (light or mm-wave/THz) energy and power the chip as well as transmit modulated signals in the waveguide. For example, the first chip may be configured to modulate the radio frequency radiation that is received and communicate neural information (e.g., by backscatter communication) for transmission through the waveguide for receipt by an external receiver. In an example embodiment, the second chip comprises an electrode site exposed along the shank surface for receiving neural information. In another example, a single chip performs both the tasks of sensing neural information from the brain as well as creation of the signal in the waveguide.

In an example embodiment, the shank is configured to have discrete, separate chipsets for preserving the flexibility of the shank, as opposed to a monolithic chipset which is relatively less flexible than an array of discrete separate chipsets where the material within which the chipsets are embedded permits movement greater than that allowed by the chipset itself.

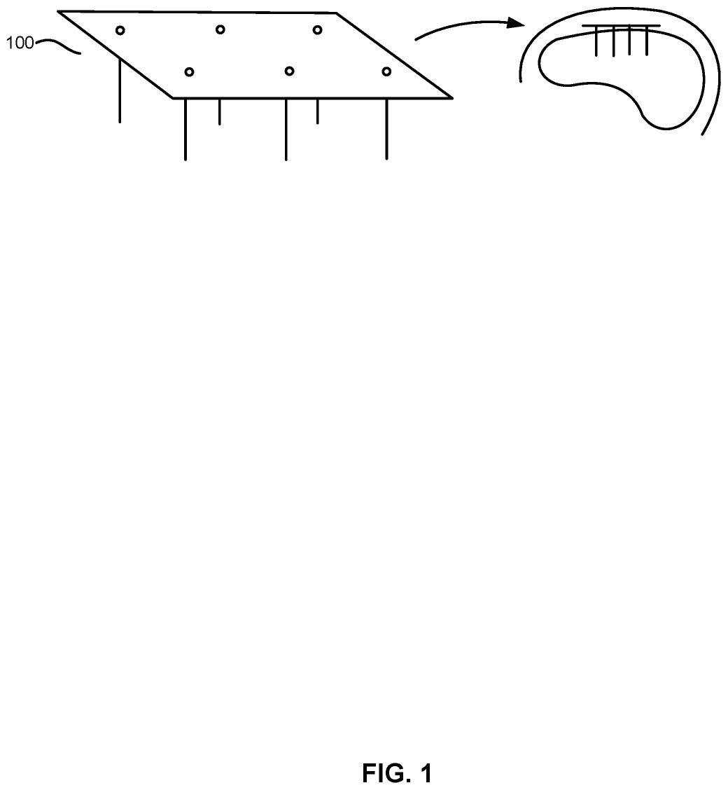

With reference now to FIG. 1, a flexible implantable electrode array 100 is illustrated. The array comprises a plurality of shanks attached to a backplane. Also illustrated is the array 100 implanted in a human brain to measure neural signals in the brain.

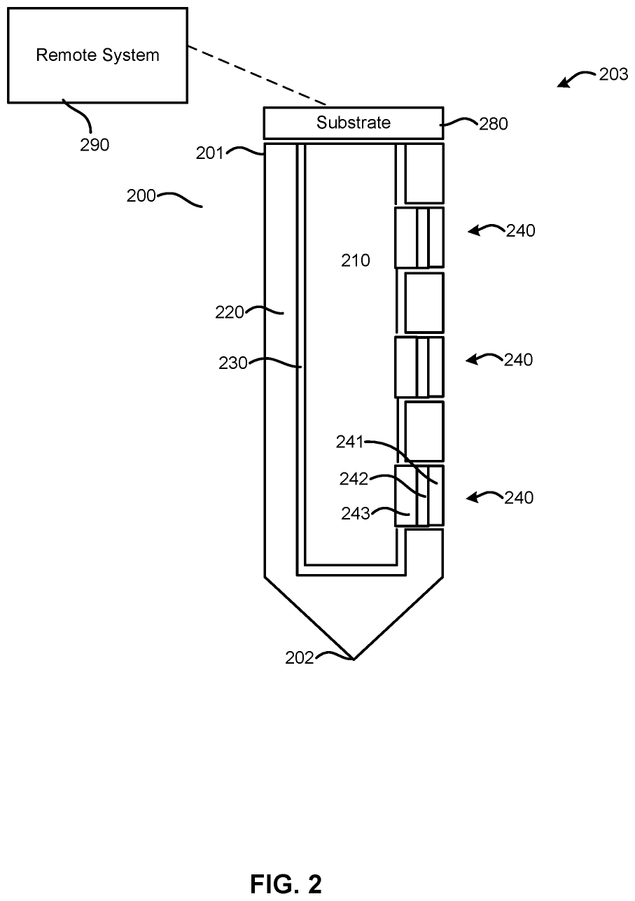

With reference now to FIG. 2, in accordance with various example embodiments, a flexible implantable electrode array 203 may comprise: a backplane 280 and a shank 200. In an example embodiment, the flexible implantable electrode array 203 may further comprise a plurality of shanks. Each of the plurality of shanks may be connected to the backplane to form an array of flexible implantable electrodes or ‘shanks.’ In various embodiments, the implantable electrode array 203 may be configured to communicate with a remote system 290.

In accordance with an example embodiment, the backplane 280 may be configured to communicate with the shank 200 and with a remote system 290. In an example embodiment, the backplane comprises a first side and a second side opposite the first side. The first side may be configured to face away from the shank, and the second side may be configured to face toward the shank (or towards a plurality of shanks attached to the second side).

In an example embodiment, the backplane 280 may be configured to communicate wirelessly with each shank 200 by transmitting and receiving signals between the backplane and a waveguide 210 contained within shank 200. In this regard, the backplane 280 may comprise a probe extending from the second side of backplane 280 into the waveguide for transmitting and/or receiving wireless signals. Alternatively, in other example embodiments, each chipset may be configured to compress the recorded data and send it serially over a wired interface bus via wires patterned on or below the shank surface. More generally, the backplane may be configured to communicate via wire connection with an antenna in the shank 200, and the antenna in the shank may communicate via the waveguide with the chipsets in the shank. In another example embodiment, the backplane 280 may be any structure that is configured to facilitate intermediary communication between the shank and a remote system. The backplane may comprise a plurality of feed horns, probes, or any devices suitable for transceiving signals to/from the waveguide.

In accordance with various example embodiments, the system may employ one or more of the following: (a) any device for launching an RF signal (e.g., mm-wave/THz signal) into the waveguide to be received by the chipsets; (b) any device for providing power through the waveguide to the chipsets; and/or (c) any device for receiving a modulated signal from the chipsets via the waveguide. Each such embodiment may have the device located in or coupled to: (1) the backplane, or (2) a location in the shank. This second embodiment may involve wired communication to the location in the shank. The location in the shank may be near the proximal end of the shank. The location may be configured to expose the device to the waveguide. In various example embodiments, the device located in the shank may be connected by wire to other devices external to the shank.

In accordance with various example embodiments, the mm-wave/THz signals may be launched into the shank(s) by means of a directional antenna that couples radiation into the waveguide. For example, the signal may be launched by a feed horn formed in backplane 280. In another example embodiment, red/infrared light may be launched into the waveguide from a light source on the backplane. In yet another example embodiment, the mm-wave/THz radiation is created by excitation of patch antennas. In another example embodiment, the modulated mm-wave/THz signal is received by a directional antenna through an opening in the backplane or an antenna mounted on top of the shank. In yet another embodiment, the modulated mm-wave/THz radiation is received by a patch antenna.

In another example embodiment, the antenna may comprise a dual-polarized antenna configured to receive on one polarization (using a port excited by this polarization) and transmit (or re-radiate) on an orthogonal polarization; this transmission can either be on a second port (exciting the orthogonal polarization) on the same antenna, or by a different antenna that is connected to the chipset. The received signal in a first embodiment may be passed through a backscatter modulator and reradiated on the orthogonal antenna or port, i.e., with different polarization and/or frequency. The received signal in a second embodiment may be multiplied with a relatively lower frequency signal from a signal oscillator (mixer/modulator) and reradiated on the orthogonal antenna or port, i.e., with different polarization and/or frequency. Moreover, any suitable method of modulation may be used.

In accordance with an example embodiment, the signal in the waveguide may be modulated through use of various modulation techniques, including: local oscillator or backscatter modulation. In accordance with various example embodiments, the chipsets are configured to cause the signals in the waveguide, created by different chipsets, to distinguish themselves, such as, for example through different carrier frequencies (Frequency Division Multiple Access, FDMA), transmission at different times (Time Division Multiple Access, TDMA), Code Division Multiple Access (CDMA), different encoding (possibly with spectrum-spreading codes), and/or different powers (non-orthogonal multiple access). Moreover, any suitable multiple access method or transmission plan may be used.

In accordance with various example embodiments, the system may be configured to provide encoding at the chipset to enable detection and/or correction of errors, to protect the transmitted data, using any suitable method for error coding, including: Cyclic Redundancy Check, block codes, convolutional codes, polar codes, turbo codes, and the like, or combinations thereof. Moreover, any suitable system for error detection/decoding in the waveguide signals may be used.

In accordance with an example embodiment, transmission of the THz signal sent into the waveguide, and modulated signal sent back to the receiver, can be on orthogonal polarization. In these example embodiments, the antennas to transmit/receive these two orthogonal polarizations can be a single patch with different feeds for the different polarizations, or it can be different antennas on different sides of the core. In various example embodiments, the antennas can be part of the chip, or just antennas, with transmission lines from the chip to the antennas. An alternative implementation has two waveguides, for example, two cores, each surrounded with metal, next to each other, with the energy signal and unmodulated THz wave transmitted in one of the two waveguides, and the modulated signal sent back in the other waveguide.

Moreover, the backplane may comprise any suitable structure for launching a signal into the waveguide 210 or receiving a signal from the waveguide 210. In an example embodiment, the backplane 280 may have a plurality of shanks 200 attached to the second side, and the 203 may be configured with a plurality of probes respectively aligned with the waveguide of each shank and configured to communicate between each shank 200 and the backplane 280. In other example embodiments (not shown), power and/or signals may be communicated by wire through the flexible shank.

In accordance with an example embodiment, the backplane is configured to communicate with the shank at gigahertz to terahertz frequency levels. For example, the backplane may be configured to communicate with the shank at 30 GHz to 1 THz frequency; and in an example embodiment in a range of 120 GHz to 300 GHz, and preferably 120 GHz to 250 GHz. Moreover, any suitable frequency range may be used.

With respect to the communications with the remote system 290, the backplane may further be configured to communicate wirelessly with the remote system 290. In another embodiment, the backplane may communicate with the remote unit via wires. In yet another embodiment, the remote unit may be physically attached to the backplane. In an example embodiment, the backplane comprises processors, memory, circuitry and/or the like configured to perform the various functions described herein. For example, the backplane may be configured to receive instructions and/or signals from the remote system, and to communicate with the shank based on those instructions and/or signals. The shanks may each be individually identifiable and thus, the remote system may cause the backplane 280 to communicate instructions to a specific one of the array of shanks. Moreover, as described below, the remote system may cause the backplane 280 to communicate with a specific one of a plurality of chipsets located along the specifically identified shank.

The backplane may further be configured to receive signals from the shank. The backplane may receive signals from each of the plurality of chipsets in the shank. The backplane may further be configured to pass those signals along to the remote system 290. Moreover, in an example embodiment, the backplane 280 is configured to process the signals received from the chipsets/shank(s) and to pass along information derived from said signals received from the chipsets/shank(s) to the remote system 290. In one example embodiment, the backplane 280 comprises transceiver(s) for communicating with the remote system 290. For example, the transceiver may be configured to communicate wirelessly. In this regard, when the 203 is implanted inside the skull, it can communicate wirelessly with the remote system 290. In an example embodiment, the first side of the backplane 280 comprises circuitry and components associated with communications with the remote system 290 and the second side of the backplane 280 comprises circuitry and components associated with communications with the shank(s).

In an example embodiment, the backplane 280 may be a printed wiring board, or any suitable backplane configured to hold circuits for the purposes described herein. In another example embodiment, the backplane is configured to be powered wirelessly. However, the backplane could be powered by a battery in another example embodiment.

With further reference to FIG. 2, the shank 200 may comprise a waveguide 210 and a chipset 240 in communication with the waveguide 210, wherein the shank is formed from a flexible material. In an example embodiment, the shank 200 has a proximal end 201 located proximate the backplane 280, and a distal end 202 opposite the proximal end 201 and distal to the backplane 280. In an example embodiment, the shank 200 is perpendicular to the backplane 280. However, the shank 200 may be at other angles to the backplane 280. In an example embodiment, the waveguide 210 extends along at least a portion of the length from the proximal end 201 to the distal end 202. Moreover, the shank 200 may comprise at least one chipset 240 (and in another example embodiment, at least two chipsets 240) located between the proximal end 201 and the distal end 202. In one example embodiment, a line of chipsets 240 may be located on the side of the shank 200, as shown in FIG. 2. In another example embodiment, not shown, two lines of chipsets 240 may be located with one on each side of the shank 200, opposing each other. In another example embodiment (not shown), the chipsets 240 may be located in various location around the shank and at various positions along the length of the shank 200.

In an example embodiment, the shank 200 comprises an outer-layer 220, a waveguide 210, and a waveguide liner 230.

In accordance with an example embodiment, the outer-layer 220 (or sheath) is configured to surround the core or waveguide 210. The outer-layer 220 is configured to provide the bulk of the structural support for the shank 200. In an example embodiment, the outer-layer 220 is configured to be flexible. The outer-layer 220 is configured to be flexible enough to reduce or minimize the damage to tissue when the shank 200 is introduced into the tissue. In an example embodiment, the outer-layer 220 comprises a flexible material. In an example embodiment, the outer-layer 220 comprises a flexible polymer material. In an example embodiment, the outer-layer 220 comprises a flexible polymer material comprising Parylene C. Other example polymer materials include polyimide, high-density polyethylene, polystyrene, and SU-8 and the like. Moreover, the flexible material may comprise any suitable material for making the shank flexible.

In an example embodiment, the waveguide 210 or ‘core’ may be an air wave guide. In other example embodiments, the waveguide 210 is filled with a flexible polymer material. Moreover, the waveguide 210 may be filled with any suitable dielectric material. In an example embodiment, the waveguide 210 is a cylindrical waveguide. The waveguide 210 may have a substantially rectangular cross-sectional shape, circular cross-sectional shape, and/or the like. In an example embodiment, the waveguide 210 may have a similar cross-sectional profile, for the entire length of the waveguide as it extends from the proximal end 201 in the direction toward the distal end 202. In other example embodiments, the cross-sectional profile may change along the length of the waveguide from proximal end 201 in the direction of the distal end 202. In this regard, changes in the cross-sectional profile may be configured to filter various frequencies, such as to differentiate frequencies assigned for communication with the respective chipsets 240. In an example embodiment, the waveguide is a dielectric waveguide.

In one example embodiment, the waveguide liner 230 is configured to define the waveguide. The waveguide liner is also described herein as a waveguide material. In an example embodiment, the waveguide material (or liner) is interposed between the core (or waveguide 210) and the layer (outer-layer 220) surrounding the core (or waveguide 210). In an example embodiment, the waveguide liner 230 extends from the proximal end 201 along at least a portion of the length of the shank in the direction of the distal end 202. In an example embodiment, the waveguide material is a metallic material. For example, in one embodiment, the waveguide material is gold. In another example embodiment, the waveguide material is platinum. In yet another example embodiment, the waveguide material is copper. Moreover, the waveguide material may be any suitable material for containing the RF signals within the waveguide.

In other example embodiments, the outer-layer 220 may be configured to define the waveguide without the need of a waveguide liner 230. For example, the outer-layer 220 may comprise a polymer-metallic blend that suitable defines the waveguide. In other example embodiments, the differences between the material filling waveguide 210 and the material of the outer-layer 220 function to define the waveguide.

In an example embodiment, the shank 200 comprises a piercing end at the distal end 202 of the shank 200. The piercing end may have a needle-like or sharp point configured to pierce into the subject tissue for facilitating insertion of the shank 200 into the subject, e.g., for piercing into the brain tissue.

In accordance with various example embodiments, the shank has an outer cross-sectional shape that is circular, rectangular, or any suitable cross-sectional shape. In an example embodiment, the largest dimension of the outer cross-sectional shape of the shank 200 (whether that be a diameter of a circular cross-section, the diagonal of a rectangle, or other representation of the largest dimension of the cross-sectional shape), is between 0.9 to 1.3 mm in outer diameter. Moreover, the largest dimension of the outer cross-sectional shape of the shank 200 can be any suitable dimension that facilitates insertion into the tissue of the test subject with reduced or minimal trauma to the subject tissue.

Accordingly, the waveguide 210 may have a largest dimension of the waveguide cross-sectional shape that is between 0.5×0.5 to 1×1 mm² if possessing a square cross section or 0.6 to 1.2 mm in outer diameter if possessing a circular cross section. In this regard, it may be advantageous to utilize terahertz frequencies for communicating through the waveguide 210 with the chipsets 240. The waveguide may be configured for communicating, for example, at frequencies between 120 to 250 GHz frequency. Moreover, any suitable frequency band may be used for communicating via the waveguide 210.

As recited above, the shank 200 may comprise one or more chipsets 240 disposed along the length of the shank 200 from the proximal end 201 toward the distal end 202. In some example embodiments, two or more chipsets 240 may be disposed along the length of the shank. In an example embodiment, the chipsets 240 may comprise a chipset electrode 241, a chipset support structure 242 (or interposer) and a chipset transceiver 243.

In an example embodiment, for each chipset 240, the chipset may be located in a gap or hole in the side of the shank 200 such that it extends from the waveguide 210 to an exterior portion of shank 200. Thus, a number of chipsets may be disposed in the shank and along the length of the shank. Stated another way, each chipset is configured to be in communication with the waveguide 210 on an interior side of chipset 240 and to be in communication with the tissue surrounding an inserted shank on an exterior side of chipset 240. In this regard, each chipset may be oriented with a chipset electrode 241 separated from a chipset transceiver 243 by a chipset support structure 242. Stated another way, the chipset electrode 241 may be mounted on a first surface of chipset support structure 242, and the chipset transceiver 243 may be mounted on a second surface of chipset support structure 242 opposite the first surface of chipset support structure 242. In an example embodiment, each of the chipsets in the shank, comprises a chipset electrode 241 that is attached back-to-back with a chipset transceiver 243. In an example embodiment, one or more of the chipset electrode 241 comprise an electrode site that is exposed to tissue external to an embedded shank. In another embodiment, a chipset might be located anywhere in the sheath, and connected electrically to one or two antennas located at the interface between core and sheath, in gaps of the metallic cladding.

In an example embodiment, each chipset 240 is configured to be powered wirelessly. For example, the chipset may be powered wirelessly from a light source external from the shank and passed to the chipsets through the waveguide. In another example embodiment, the chipset is powered wirelessly via radio frequency radiation from a radiation source external to the shank, such as with RF power harvesting. In various example embodiments the RF radiation is also passed to the chipsets through the waveguide. In accordance with various example embodiments, the chipsets are configured to receive light in a red wavelength of the light spectrum (e.g., 750 nm wavelength). Moreover, any suitable light wavelength may be used. In another embodiment, the chipset is powered by mm-wave/THz electromagnetic waves whose frequency may or may not coincide with the frequency of the waves that are modulated by the chipsets.

In an example embodiment, a chipset of the plurality of chipsets may comprise complementary metal oxide semiconductor (CMOS) integrated circuits for power harvesting, radio frequency backscatter communication, and multiplexing, amplifying, and/or recording neuron signals.

In an example embodiment, the chipset electrode 241 is configured to receive neural signals. Stated another way, the chipset electrode 241 is configured to passively monitor neural activity in cells proximate the chipset electrode 241. In another example embodiment, the chipset electrode 241 is configured to transmit and receive. For example, the chipset electrode 241 may be configured to provide a stimulus signal to the tissue surrounding the chipset electrode 241, and to monitor the neural response of the stimulated tissue/cells. In an example embodiment, the chipset electrode 241 is configured for multiplexed neural recording. Stated another way, the chipset electrode 241 may comprise an electrode site exposed (to the brain tissue in a manner that it can receive neural signals from the brain tissue) along the shank surface and the chipset electrode 241 is positioned opposite the chipset transceiver 243 and comprises the electrode site. The chipset electrode 241 may comprise an array of on-chip electrodes, e.g. 256 electrodes in a 16×16 array.

In an example embodiment, the chipset transceiver 243 is configured to receive signals and/or power from the backplane 280. In this regard, the chipset transceiver 243 may comprise a probe or other waveguide receiver for receiving signals and/or power communicated via the waveguide. In another example embodiment, the chipset transceiver 243 is configured to transmit signals responsive to the received signals and/or responsive to the signals received from the chipset electrode 241. Thus, the chipset transceiver 243 may be configured for both power harvesting and radio frequency communication with the backplane 280. In an example embodiment, the chipset transceiver 243 may further comprise a photodiode to rectify the visible red light for power harvesting, to wirelessly power the chipset. In an example embodiment, the chipset transceiver 243 is positioned within the shank outside of the core and adjacent the waveguide.

In accordance with various example embodiments, the chipset electrode 241 may communicate with the chipset transceiver 243. For example, the chipset transceiver 243 may provide control signals and/or power from the chipset transceiver 243 to the chipset electrode 241. Moreover, the chipset electrode 241 may provide data/signals based on the electrode measurements at chipset electrode 241.

In an example embodiment, chipset electrode 241 and/or chipset transceiver 243 may be configured to perform various data processing actions on the signal received at the electrode. For example, the data processing actions may comprise quantization of the signals from the neurons, data compression, error correction and/or error detection coding, and/or mapping to symbols that are the input for the modulator. As noted above, the chipset electrode 241 and chipset transceiver 243 may be located on opposing sides of a printed wiring board or other separator/support structure. In this example embodiment, the chipset electrode 241 may communicate with the chipset transceiver 243 via the chipset support structure 242.

In accordance with various example embodiments, the remote system 290 may comprise a server, a processor, a database and or the like. In one example embodiment, the remote system is at least partially in the cloud. In an example embodiment, the remote system is configured to communicate with backplane 280. For example, the remote system 290 may be configured to send commands to backplane 280 to cause backplane 280 to send signals to the chipsets 240, to power chipsets 240, to receive signals from chipsets 240, to process information received from chipsets 240, and/or to send signals back to remote system 290. In another example, the remote system 290 may be configured to receive signals from backplane 280. The received signals may include data indicative of the responses to the commands sent from remote system 290, neural signals received from each chipset, and/or the like. In an example embodiment, remote system 290 is configured to communicate wirelessly with backplane 280. In this example embodiment, remote system 290 may comprise a wireless transmitter and/or receiver. Moreover, any suitable method of communicating with backplane 280 may be used, e.g., Bluetooth, wired, etc.

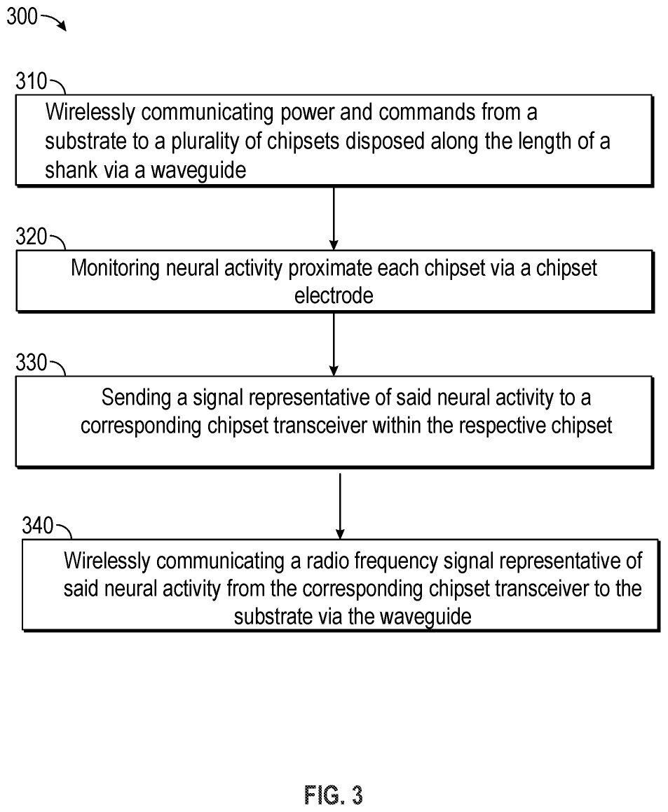

In accordance with various example embodiments, and with reference to FIG. 3, a method 300 for powering and receiving neuronal information from a flexible implantable electrode array is disclosed. The method 300 may comprise wirelessly communicating power and commands from a backplane to a plurality of chipsets disposed along the length of a shank via a waveguide disposed within the shank, wherein the shank is formed form a flexible polymer material (310). The method 300 may further comprise monitoring neural activity proximate each chipset via a chipset electrode (320), and sending a signal representative of said neural activity to a corresponding chipset transceiver within the respective chipset (330). The method 300 may further comprise wirelessly communicating a radio frequency signal representative of said neural activity from the corresponding chipset transceiver to the backplane via the waveguide (340). The method 300 may further comprise wirelessly providing power to each chipset from the backplane via the waveguide.

The method 300 may further comprise gathering neuronal information from one or more of the chipsets, wherein the one or more chipsets comprise an electrode site that is exposed along a surface of the shank for contacting a portion of a brain. The method may further comprise modulating the radio frequency signal to differentiate the signals from one of the number of chipsets from the other chipsets.

Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted except in light of the appended claims and their equivalents.

What is claimed is:

1. A flexible implantable electrode array comprising:
a shank formed from a flexible polymer material, the shank comprising:
a waveguide; and
a number of chipsets disposed in the shank along the length of the shank, wherein each chipset is configured to measure neural activity in tissue surrounding the shank near the respective chipset, and to communicate signals representative of the measured neural activity via the waveguide.

2. The flexible implantable electrode array as recited in claim 1, wherein the flexible polymer material is Parylene C.

3. The flexible implantable electrode array as recited in claim 1, further comprising a backplane, and wherein:
the shank is attached to the backplane;
the chipsets are configured to be powered wirelessly from the backplane;
the chipsets are configured to receive commands from the backplane; and
the chipsets are configured to provide a data signal to the backplane via the waveguide, the data signal representative of the measured neural activity.

4. The flexible implantable electrode array as recited in claim 1, wherein the shank comprises the waveguide and an outer-layer surrounding the waveguide.

5. The flexible implantable electrode array as recited in claim 4, wherein a waveguide material is interposed between the waveguide and the outer-layer surrounding the waveguide.

6. The flexible implantable electrode array as recited in claim 5, wherein the waveguide material is formed from a metallic material.

7. The flexible implantable electrode array as recited in claim 6, wherein the metallic material is gold.

8. The flexible implantable electrode array as recited in claim 1, wherein one or more of the number of chipsets comprises complementary metal oxide semiconductor (CMOS) integrated circuits for power harvesting, radio frequency backscatter communication, and multiplexing, amplifying, and recording neuron signals.

9. The flexible implantable electrode array as recited in claim 7, wherein one or more of the number of chipsets comprises a chipset transceiver that is attached back-to-back with a chipset electrode.

10. The flexible implantable electrode array as recited in claim 1, wherein one or more of the number of chipsets comprise an electrode site that is exposed along the shank.

11. A flexible implantable electrode array for receiving and communicating neuronal information comprising:
a shank formed from a flexible polymer material and having a length greater than a width, wherein the shank comprises a waveguide and a sheath surrounding the waveguide;
a waveguide liner disposed in the shank and interposed between the waveguide and the sheath, the waveguide liner formed from a metallic material and extending along a length of the waveguide; and
an array of chipsets comprising a number of chipsets positioned serially along the length of the shank, wherein each chipset includes an electrode site that is exposed along a surface of the shank, wherein each chipset communicates signals measured at the electrode site via the waveguide.

12. The flexible implantable electrode array as recited in claim 11, wherein the polymer shank is formed from Parylene C.

13. The flexible implantable electrode array as recited in claim 11, wherein each chipset is configured to be powered wirelessly via the waveguide.

14. The flexible implantable electrode array as recited in claim 11, the number of chipsets comprises complementary metal oxide semiconductor (CMOS) integrated circuits for power harvesting, radio frequency backscatter communication, and multiplexing, amplifying, and recording neuron signals.

15. The flexible implantable electrode array as recited in claim 14, wherein one or more of the number of chipsets comprises a chipset transceiver for power harvesting and radio frequency communication that is attached back-to-back with a chipset electrode for multiplexed neural recording.

16. The flexible implantable electrode array as recited in claim 15, wherein the chipset transceiver is positioned within the shank outside of and adjacent to the waveguide, and the chipset electrode is positioned opposite the chipset transceiver and comprises the electrode site.

17. A method for powering and receiving neuronal information from a flexible implantable electrode array comprising:
wirelessly communicating power and commands from a backplane to a plurality of chipsets disposed along the length of a shank via a waveguide disposed within the shank, wherein the shank is formed form a flexible polymer material;
monitoring neural activity proximate each chipset via a chipset electrode, and sending a signal representative of said neural activity to a corresponding chipset transceiver within the respective chipset; and
wirelessly communicating a radio frequency signal representative of said neural activity from the corresponding chipset transceiver to the backplane via the waveguide.

18. The method of claim 17, further comprising wirelessly providing power to each chipset from the backplane via the waveguide.

19. The method of claim 18, wherein the power is provided via light transmitted through the waveguide.

20. The method of claim 17, further comprising gathering neuronal information from one or more of the chipsets, wherein the one or more chipsets comprise an electrode site that is exposed along a surface of the shank for contacting a portion of a brain.
